(19) United States
(12) Patent Application Publication
(10) Pub. No.: US 2017/0156851 A1
(43) Pub. Date: Jun. 8, 2017

(54) IRRIGATING INTRAOCULAR LENS ROTATORS AND RELATED METHODS

(71) Applicant: Arvind Saini, Temecula, CA (US)

(72) Inventor: Arvind Saini, Temecula, CA (US)

(21) Appl. No.: 15/432,703

(22) Filed: Feb. 14, 2017

Related U.S. Application Data
(63) Continuation of application No. 14/257,629, filed on Apr. 21, 2014.

(51) Int. Cl. A61F 2/16; A61F 9/007

(52) U.S. Cl. CPC A61F 2/1662; A61F 9/00736; A61F 2002/1681

(57) ABSTRACT

Apparatus for positioning an intraocular lens (IOL) having a haptic within the capsular bag of an eye. The apparatus comprises a handle portion having a proximal end, a distal end, and an irrigation lumen extending between the proximal end and the distal end. The apparatus further comprises a lens engagement portion at the distal end of the handle portion. The lens engagement portion includes a superior surface defining a haptic engagement surface configured to engage the haptic of the IOL, and a plurality of irrigation ports arranged about the haptic engagement surface. Each of the irrigation ports is in fluid communication with the irrigation lumen.

### BACKGROUND

The present embodiments relate to devices and methods for rotating intraocular lenses.

Cataract surgery is a procedure for removing a cloudy lens from the eye. Usually, an intraocular lens (IOL) is implanted at the same time. The IOL is implanted within the capsular bag inside the eye. The capsular bag is a sack-like structure remaining within the eye following extracapsular cataract extraction or phacoemulsification. The implanted IOL is placed within this structure to recreate the usual phakic state.

With reference to FIG. 1, a typical IOL 100 includes a small plastic lens 102 with side struts, called haptics 104, that hold the lens 102 in place within the capsular bag inside the eye. After implantation, the lens 102 must be rotated into the correct orientation to work properly, particularly in the case of toric lenses. A toric lens is a lens with different optical power and focal length in two orientations perpendicular to each other. Rotating the lens 102 is a delicate procedure, as the capsular bag is very thin, as thin as 5 microns at the posterior pole, and tearing the capsular bag can lead to complications and a lengthening of the procedure.

### SUMMARY

The various embodiments of the present irrigating intraocular lens rotators and related methods have several features, no single one of which is solely responsible for their desirable attributes. Without limiting the scope of the present embodiments as expressed by the claims that follow, their more prominent features now will be discussed briefly. After considering this discussion, and particularly after reading the section entitled “Detailed Description,” one will understand how the features of the present embodiments provide the advantages described herein.

One of the present embodiments comprises apparatus for positioning an intraocular lens (IOL) having a haptic within the capsular bag of an eye. The apparatus comprises a handle portion having a proximal end, a distal end, and an irrigation lumen extending between the proximal end and the distal end. The apparatus further comprises a lens engagement portion at the distal end of the handle portion. The lens engagement portion includes a superior surface defining a haptic engagement surface configured to engage the haptic of the IOL, and a plurality of irrigation ports arranged about the haptic engagement surface. Each of the irrigation ports is in fluid communication with the irrigation lumen.

Another of the present embodiments comprises apparatus for positioning an intraocular lens (IOL) having a haptic within the capsular bag of an eye. The apparatus comprises a tubular sleeve including a distal end. The sleeve is configured to receive a distal tip portion of an IOL installation handpiece having an irrigation lumen. The apparatus further comprises a lens engagement portion at the distal end of the sleeve. The lens engagement portion includes a superior surface defining a haptic engagement surface configured to engage the IOL. The haptic engagement surface includes a plurality of irrigation ports arranged about the haptic engagement surface. When the sleeve is secured to the IOL installation handpiece, the irrigation ports are in fluid communication with the irrigation lumen.

### BRIEF DESCRIPTION OF THE DRAWINGS

The various embodiments of the present irrigating intraocular lens rotators and related methods now will be discussed in detail with an emphasis on highlighting the advantageous features. These embodiments depict the novel and non-obvious irrigating intraocular lens rotators and related methods shown in the accompanying drawings, which are for illustrative purposes only. These drawings include the following figures, in which like numerals indicate like parts:

FIG. 1 is a side perspective view of a typical intraocular lens (IOL);

FIG. 2 is a side elevation view of an irrigating IOL rotator according to the present embodiments;

FIG. 3 is a top plan view of the irrigating IOL rotator of FIG. 2;

FIGS. 4A and 4B are front elevation views of alternative profiles for the distal end of the irrigating IOL rotator of FIG. 2, as viewed along the line 4A/4B in FIG. 2;

FIG. 5 is a partial cross-sectional side elevation view of another irrigating IOL rotator according to the present embodiments;

FIG. 6 is a side perspective view of the irrigating IOL rotator of FIG. 5 engaged with an IOL;

FIG. 7 is a side elevation view of a prior art irrigation/aspiration probe;

FIG. 8 is a side elevation view of a sleeve configured for use with an irrigation/aspiration probe according to the present embodiments;

FIG. 9 is a front elevation view of the sleeve of FIG. 8;

FIG. 10 is a side elevation view of the sleeve of FIG. 8 positioned on the irrigation/aspiration probe of FIG. 7;

FIG. 11 is a partial cross-sectional side elevation view of another sleeve configured for use with an irrigation/aspiration probe according to the present embodiments;

FIG. 12 is a partial cross-sectional side elevation view of another sleeve configured for use with an irrigation/aspiration probe according to the present embodiments;

FIG. 13 is a side elevation view of a prior art bimanual irrigation probe;

FIG. 14 is a cross-sectional side elevation view of a sleeve configured for use with a bimanual irrigation probe according to the present embodiments;

FIG. 15 is a cross-sectional side elevation view of another irrigating IOL rotator according to the present embodiments;

FIG. 16 is a front elevation view of the irrigating IOL rotator of FIG. 15;

FIG. 17 is a side perspective view of the irrigating IOL rotator of FIG. 15 engaged with an IOL;

FIG. 18A is a front elevation view of another irrigating IOL rotator according to the present embodiments;

FIG. 18B is a top plan view of the irrigating IOL rotator of FIG. 18A;

FIG. 19A is a front elevation view of another irrigating IOL rotator according to the present embodiments;

FIG. 19B is a top plan view of the irrigating IOL rotator of FIG. 19A; and

FIG. 20 is a cross-sectional side elevation view of another irrigating IOL rotator according to the present embodiments.

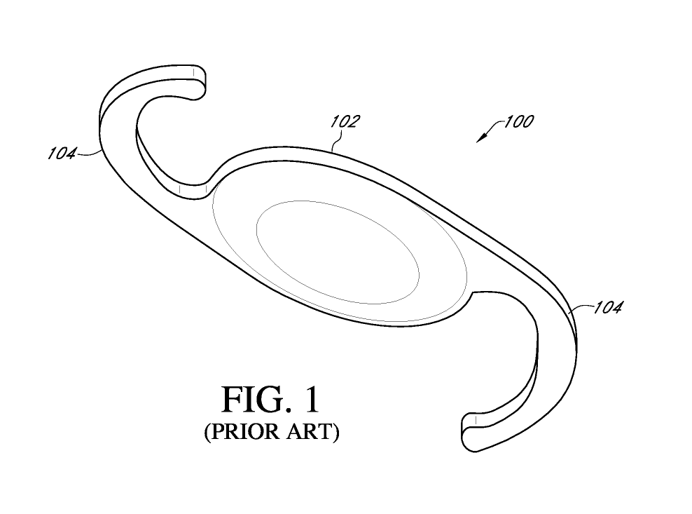

### DETAILED DESCRIPTION

The following detailed description describes the present embodiments with reference to the drawings. In the drawings, reference numbers label elements of the present embodiments. These reference numbers are reproduced below in connection with the discussion of the corresponding drawing features.

The embodiments of the present irrigating intraocular lens rotators and related methods are described below with reference to the figures. These figures, and their written descriptions, indicate that certain components of the apparatus are formed integrally, and certain other components are formed as separate pieces. Those of ordinary skill in the art will appreciate that components shown and described herein as being formed integrally may in alternative embodiments be formed as separate pieces. Those of ordinary skill in the art will further appreciate that components shown and described herein as being formed as separate pieces may in alternative embodiments be formed integrally. Further, as used herein the term integral describes a single unitary piece.

The present embodiments include methods of using an irrigating intraocular lens rotator. Some of these embodiments may be performed in connection with treating a human and/or animal body. Others of these embodiments may be performed independently of a human and/or animal body, such as for purposes of testing or demonstration of the irrigating intraocular lens rotator. Accordingly, the present embodiments pertaining to methods of using an irrigating intraocular lens rotator should not be construed as limited to methods of treating a human and/or animal body.

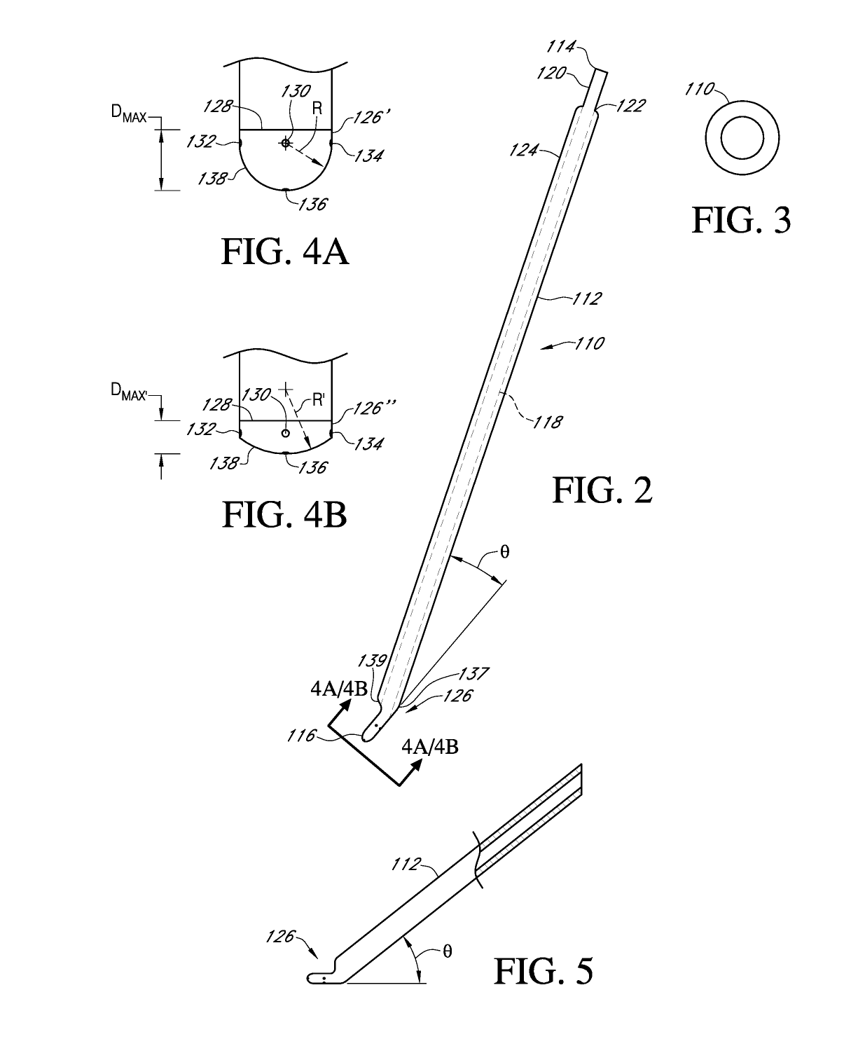

FIGS. 2 and 3 illustrate one embodiment of apparatus 110 for positioning an intraocular lens (IOL) within the capsular bag of an eye. With reference to FIG. 2, the apparatus 110, which may also be referred to interchangeably as a handpiece 110, comprises an elongate, tubular handle portion 112 having a proximal end 114 and a distal end 116. An irrigation lumen 118 extends between the proximal end 114 and the distal end 116. The proximal end 114 includes a reduced diameter portion 120 and an external shoulder 122 at a junction of the reduced diameter portion 120 and a main body portion 124 of the handpiece 110. The proximal end 114 is configured to receive standard irrigation tubing (not shown) of the type commonly used in procedures for cataract extraction. For example, the irrigation tubing may be slid over the reduced diameter portion 120 and held in place with a friction fit. Additional structure, such as one or more ribs, lips, threads, etc., may be provided on one or both of the handpiece 110 and the irrigation tubing to further secure the tubing on the handpiece 110. As described in further detail below, irrigation fluid supplied through the irrigation tubing flows through the irrigation lumen 118 and is expelled through the distal end 116 of the handpiece 110. In alternative embodiments (not shown), the proximal end of the handpiece may be configured to receive irrigation tubing with the tubing being inserted into the handpiece, rather than being slid over the handpiece.

The distal end 116 of the handle portion 112 includes a lens engagement portion 126. FIGS. 4A and 4B illustrate two alternative configurations of the lens engagement portion 126′, 126″. With reference to both FIGS. 4A and 4B, the lens engagement portion 126 includes a flat superior surface defining a haptic engagement surface 128. The haptic engagement surface 128 is configured to engage the IOL, particularly the haptic(s) of the IOL. For example, the haptic engagement surface 128 may be positioned beneath a haptic of an IOL during a procedure to implant the IOL within the capsular bag, as described in further detail below. In various embodiments, the haptic engagement surface 128 may be smooth or textured to provide a desired amount of friction between the haptic engagement surface 128 and the haptic. For example, texturing may be provided on the haptic engagement surface 128 by knurling, sand blasting, or any other suitable process.

With further reference to FIGS. 4A and 4B, a plurality of irrigation ports 130, 132, 134, 136 are arranged about the haptic engagement surface 128. The irrigation ports 130, 132, 134, 136 are in fluid communication with the irrigation lumen 118 such that the irrigation fluid flowing through the irrigation lumen 118 is expelled through the irrigation ports 130, 132, 134, 136. As described further below, during an IOL implantation procedure irrigating fluid expelled through the ports 130, 132, 134, 136 pushes the capsular bag outward, out of the way of the rotating IOL and handpiece 110.

In the illustrated embodiment, four irrigation ports 130, 132, 134, 136 are provided, including an anterior irrigation port 130, a pair of lateral irrigation ports 132, 134 on opposite sides of the haptic engagement surface 128, and an inferior irrigation port 136. The illustrated configuration of the irrigation ports 130, 132, 134, 136 is just one example and is not limiting, but is nonetheless particularly advantageous. For example, at least the anterior port 130 (and to a lesser extent also the lateral ports 132, 134 and the inferior port 136) is positioned to create a distending wave of fluid ahead of the lens engagement portion 126 as it moves forward, and at least the inferior port 136 (and to a lesser extent also the lateral ports 132, 134 and the anterior port 130) is positioned to create a fluid wave directed inferior of the lens engagement portion 126 to distend the posterior capsular bag to further create a cushion of fluid around the lens engagement portion 126 and the IOL. In some embodiments, the anterior port 130 may be slightly angled with respect to the horizontal such that irrigation fluid expelled through the anterior port 130 is directed slightly downward. This flow direction may augment the fluid wave directed inferior of the lens engagement portion 126 to distend the posterior capsular bag.

In the illustrated embodiments of FIGS. 2, 3, 4A and 4B, the lens engagement portion 126 does not include an aspiration port. Thus, no aspiration is used during an IOL implantation procedure using the embodiments of FIGS. 2, 3, 4A and 4B. Avoiding aspiration provides several advantages. For example, there is no concern that suction from an aspiration port might inadvertently capture the capsular bag, which is one way that the capsular bag can be torn, as described below.

In a typical cataract extraction using phacoemulsification, the eye's internal lens is emulsified with ultrasonic vibrations from a phacoemulsification probe (also referred to as a handpiece) and then aspirated from the eye. The phacoemulsification probe is typically controlled with a foot pedal switch. There are usually four distinct positions for the switch. In a first switch position in which the foot pedal is not depressed, no irrigation or aspiration takes place through the handpiece. In a second switch position in which the foot pedal is depressed lightly, irrigation fluid flows through the handpiece, but no aspiration occurs. In a third switch position in which the foot pedal is depressed more firmly, irrigation fluid and vacuum/aspiration are both applied through the handpiece. In a fourth switch position in which the foot pedal is depressed completely, phacoemulsification begins. Irrigation and/or aspiration may also occur during phacoemulsification.

After the lens is aspirated from the eye, the phacoemulsification probe is disconnected from the irrigation/aspiration (I/A) tubing, and a typical I/A handpiece is connected to the tubing. During this portion of the procedure, the same foot pedal switch described above may be used to control the I/A handpiece, but the third and fourth positions of the switch provide the same functionality, i.e. irrigation and aspiration but no phacoemulsification. That is, in the first switch position in which the foot pedal is not depressed, no irrigation or aspiration takes place through the I/A handpiece. In the second switch position in which the foot pedal is depressed lightly or half way, irrigation fluid flows through the I/A handpiece, but no aspiration occurs. In either the third or the fourth switch position in which the foot pedal is depressed more firmly, vacuum/aspiration is applied through the I/A handpiece, and irrigation also continues. An intensity or force of the aspiration and/or irrigation may be selectable by varying the force applied to the foot pedal switch.

In a typical cataract extraction and IOL implantation procedure, damage to the capsular bag can occur when the bag is inadvertently aspirated into the handpiece, which usually tears the bag and can happen in a matter of milliseconds. Certain of the present embodiments avoid the possibility of damaging the capsular bag through inadvertent aspiration by avoiding aspiration altogether. For example, as described above, the embodiments of FIGS. 2, 3, 4A, and 4B include no aspiration port, but are configured for use with typical I/A tubing and foot pedal switches (or other types of switches). Thus, a surgeon advantageously need not worry about applying too much force to the foot pedal and thereby accidentally engaging aspiration, which could damage the capsular bag. The surgeon may instead focus on properly positioning and orienting the IOL with the handpiece 110. Even if aspiration is accidentally engaged with the switch, no aspiration will take place through the handpiece 110 because no aspiration port is present. The embodiments of FIGS. 2, 3, 4A, and 4B, and others of the present embodiments, are therefore advantageous for use in positioning an IOL in most situations.

Certain others of the present embodiments may enable aspiration for those situations in which aspiration may be desired. During a cataract extraction and IOL implantation procedure, the eye is generally a closed environment. However, fluid may escape through the incision when the handpiece extends through the incision. If the diameter of the handpiece is significantly smaller than the incision, then most or all of the excess irrigating fluid will be expelled through the incision. However, if the handpiece and the incision are closely matched in size, then pressure within the eye may increase when inflowing irrigating fluid cannot escape through the incision. In this situation, aspiration may be beneficial for relieving pressure within the eye. If aspiration is used, it is beneficial to have the aspiration port located on the anterior surface of the handpiece 110, 180° away from the capsule, and preferably as proximal as possible, yet remain in the eye when used, at least because this portion of the handpiece 110 is least likely to come into close proximity with the capsule. However, the foregoing placement for the aspiration port is not limiting, as the aspiration port may be placed anywhere on the handpiece, including at an elbow bend 137 (FIG. 2) near the junction of the handle portion 112 and the lens engagement portion 126. If the aspiration port is positioned at the elbow bend 137, irrigating fluid may also be directed posteriorly to distend the capsular bag.

With continued reference to FIGS. 4A and 4B, both of the illustrated embodiments include a convex inferior surface 138. However, the convex inferior surfaces 138 include different radii. With reference to FIG. 4A, the radius R of the inferior surface 138 is shorter than the maximum perpendicular distance D_{MAX} between the inferior surface 138 and the haptic engagement surface 128. By contrast, with reference to FIG. 4B, the radius R′ of the inferior surface 138 is longer than the maximum perpendicular distance D_{MAX} ′ between the inferior surface 138 and the haptic engagement surface 128. While FIGS. 4A and 4B illustrate two example embodiments, in alternative embodiments (not shown) the maximum perpendicular distance between the inferior surface 138 and the haptic engagement surface 128 may have any value. For example, the radius of the inferior surface 138 may be equal to the maximum perpendicular distance between the inferior surface 138 and the haptic engagement surface 128. Further, in various embodiments (not shown) the inferior surface of the handpiece may not be convex, and may be, for example, flat.

With reference to FIG. 2, the handle portion 112 and the lens engagement portion 126 define an angle Θ between them. The angle Θ may have any value, such as between approximately 5° and approximately 50°. In the embodiment of FIG. 2, Θ is approximately 15°. FIG. 5 illustrates an alternative embodiment in which Θ is approximately 40°.

With further reference to FIG. 2, a distal end of the main body portion 124 includes a shoulder 139 where the main body portion 124 meets the lens engagement portion 126. The shoulder 139 provides a surface against which the IOL may bear during the IOL implantation procedure, which may aid in controlling the positioning of the IOL.

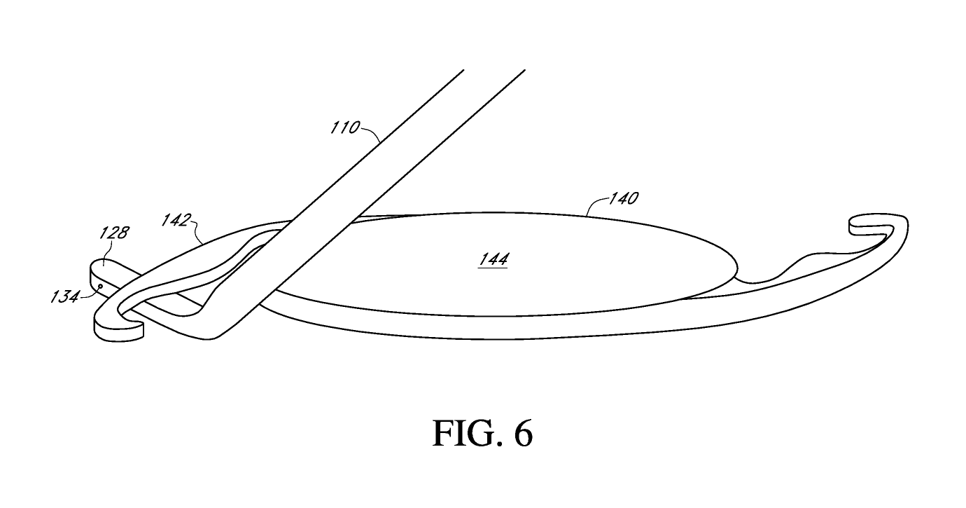

FIG. 6 illustrates the handpiece 110 of FIG. 5 engaged with an IOL 140 having a pair of haptics 142 extending from a generally circular lens portion 144. The haptic engagement surface 128 is positioned beneath and supports a first one of the haptics 142. The operator can rotate the IOL 140 within a plane defined by the lens portion 144 by gently lifting the haptic 142 from underneath and moving the handpiece 110 clockwise or counterclockwise about a center of the lens portion 144. The lifting force applied to the underside of the haptic 142 generates friction between the haptic 142 and the haptic engagement surface 128 so that the IOL 140 rotates in either direction as the operator moves the handpiece 110. The operator can continue rotating the IOL 140 in either direction until the desired rotational orientation is reached. Before, during, and/or after rotating the IOL 140, irrigating fluid may be expelled through the ports 130, 132, 134, 136 located about the haptic engagement surface 128. The irrigating fluid may act as a lubricant to facilitate rotating the IOL 140 within the capsular bag. The irrigating fluid may also push the capsular bag outward, out of the way of the rotating IOL 140 and/or handpiece 110. Also before, during, and/or after rotating the IOL 140, aspiration may be used to clear away excess irrigating fluid.

The handpieces 110 of the foregoing embodiments are preferably constructed of a rigid or semi-rigid medical grade material, such as a metal or a polymer. Example materials include, without limitation, stainless steel, titanium, acrylonitrile butadiene styrene (ABS), silicone, etc. The handpieces 110 may be single use (disposable) or reusable.

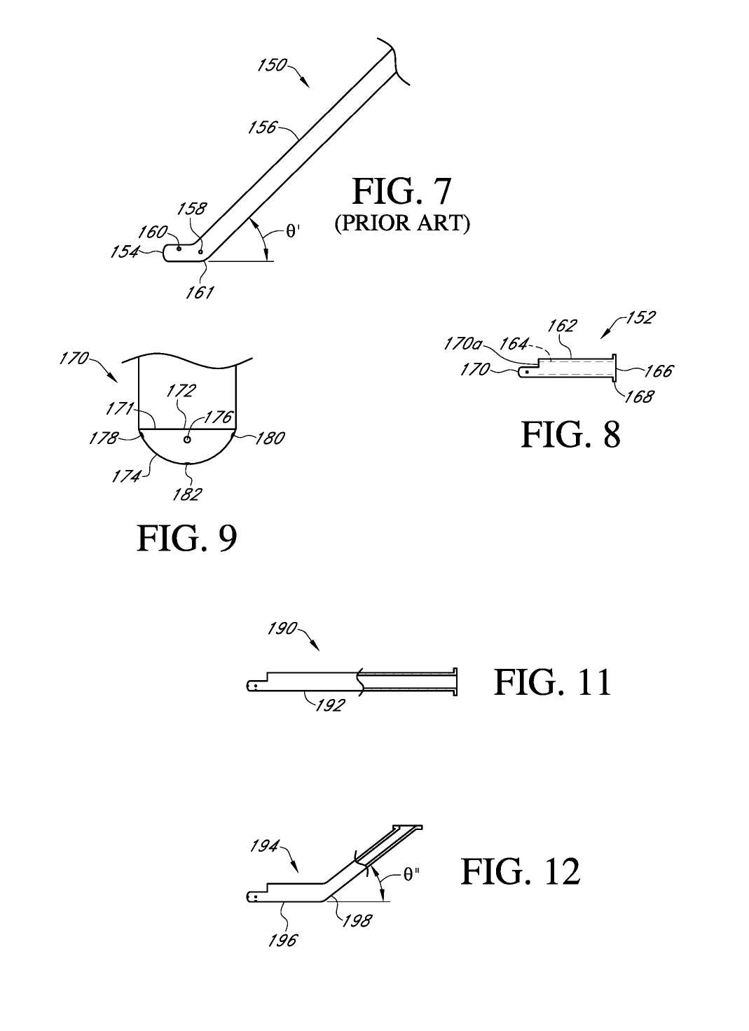

FIG. 7 illustrates a typical irrigation/aspiration probe or handpiece 150, and FIGS. 8 and 9 illustrate another of the present embodiments configured for use with the handpiece 150 of FIG. 7. The embodiment of FIGS. 8 and 9 comprises a sleeve 152 that fits over a distal tip portion 154 of the handpiece 150 of FIG. 7. With reference to FIG. 7, the handpiece 150 comprises an elongate handle portion 156 having a proximal end (not shown), with the distal tip portion 154 joined to the handle portion 156 at an angle, as described below. An irrigation port 158 and an aspiration port 160 are located in the distal tip portion 154. The irrigation port 158 is located just distal of an elbow bend 161 forming the juncture of the handle portion 156 and the distal tip portion 154, and the aspiration port 160 is located in the superior surface of the distal tip portion 154. An irrigation lumen (not shown) extends between the proximal end of the handpiece 150 and the irrigation port 158 in the distal tip portion 154. A separate aspiration lumen (not shown) extends between the proximal end of the handpiece 150 and the aspiration port 160 in the distal tip portion 154. The distal tip portion 154 and the handle portion 156 define an angle Θ′ therebetween.

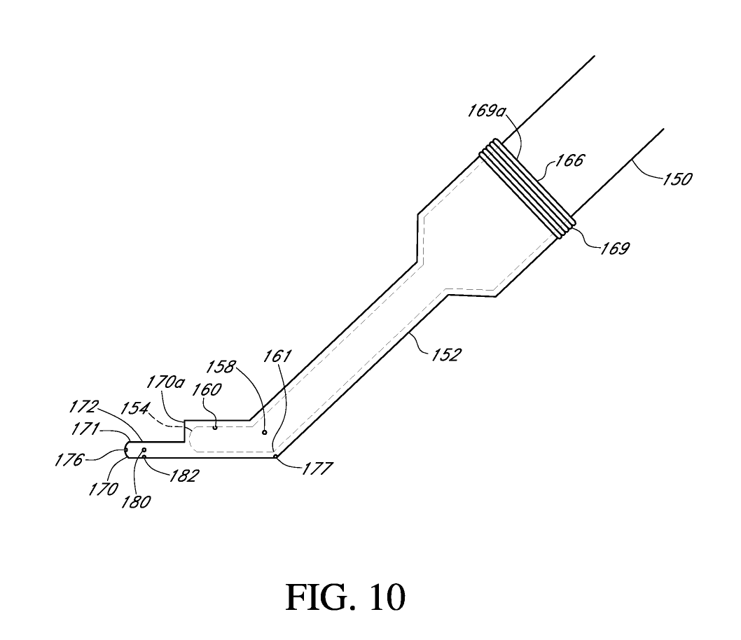

With reference to FIG. 8, the sleeve 152 includes a tubular portion 162 defining a passage 164 configured to receive the distal tip portion 154 of the handpiece 150, as shown in FIG. 10. With reference to FIGS. 8 and 10, a proximal end 166 of the sleeve 152 may include an outwardly extending flange 168 (FIG. 8) or outwardly extending ridges 169 (FIG. 10) that facilitates gripping the sleeve 152 when placing it over the handpiece 150 and/or removing it from the handpiece 150. With reference to FIG. 10, the proximal end 166 of the sleeve 152 may include inwardly extending flanges 169a that engage the handpiece 150 an increase the frictional hold of the sleeve 152 on the handpiece 150.

With reference to FIGS. 8-10, a distal end 170 of the sleeve 152 includes a shoulder 170a where the diameter of the sleeve 152 steps down. The shoulder 170a provides a barrier against which the distal tip portion 154 of the handpiece 150 bears, as shown in FIG. 10. The distal end 170 of the sleeve 152 extends beyond the distal tip portion 154 of the handpiece 150 and includes a flat superior portion 171 defining a haptic engagement surface 172. The haptic engagement surface 172 is configured to engage the IOL in a similar manner as described above with respect to the foregoing embodiments. The haptic engagement surface 172 may be smooth or textured, as described above with respect to the foregoing embodiments.

With reference to FIG. 9, the distal end 154 of the sleeve 152 further includes a convex inferior surface 174 and four irrigation ports 176, 178, 180, 182 positioned about the haptic engagement surface 172, with one anterior port 176, two lateral ports 178, 180, and one inferior port 182, as described above with respect to the foregoing embodiments. The sleeve 152 is thus configured to enable irrigation during an IOL placement procedure in a similar manner as described above with respect to the foregoing embodiments.

With reference to FIG. 10, the sleeve 152 may include a further irrigation port 177 that overlies the elbow bend 161 of handpiece 150 and provides an additional irrigation outlet for the irrigation port 158. Proximally of the shoulder 170a, a superior portion of the sleeve 152 includes no openings, and thus covers and seals the aspiration port 160 of the handpiece 150 such that no aspiration occurs with the sleeve 152 of FIGS. 8-10. However, in alternative embodiments an opening may be provided in the superior portion in a location corresponding to the aspiration port 160 of the handpiece 150 so that aspiration may occur through the alternative sleeve. In still further alternative embodiments, one or more openings may be provided anywhere in the sleeve in location(s) corresponding to aspiration port(s) of different handpieces that may have one or more aspiration ports in locations other than as illustrated in FIG. 7.

FIG. 11 illustrates another embodiment of a sleeve 190 configured for use with a typical irrigation/aspiration handpiece. The sleeve 190 of FIG. 11 is similar to the sleeve 152 of FIG. 8, except that the tubular portion 192 has a longer length.

FIG. 12 illustrates another embodiment of a sleeve 194 configured for use with a typical irrigation/aspiration handpiece. The sleeve 194 of FIG. 12 is similar to the sleeve 190 of FIG. 11, except that the tubular portion 196 includes a bend 198 defining an angle Θ″. The angle Θ″ may have any value, but in certain embodiments the value of Θ″ may be equal to the value of the angle Θ′ defined between the distal end 154 and the handle portion 156 of the handpiece 150 of FIG. 7. The sleeve 194 of FIG. 12 is thus configured to more closely match the geometry of the handpiece 150.

The sleeves 152, 190, 194 of the foregoing embodiments are preferably constructed of a flexible and resilient medical grade material, including polymers such as silicone. The sleeves 152, 190, 194 may be single use (disposable) or reusable.

While the sleeves 152, 190, 194 described above and shown in FIGS. 8-12 and 14 are configured to fit the illustrated handpieces 150, 202 shown in FIGS. 7, 10, and 13, in other embodiments one or more sleeves may be configured to fit other handpieces having different structure from that shown.

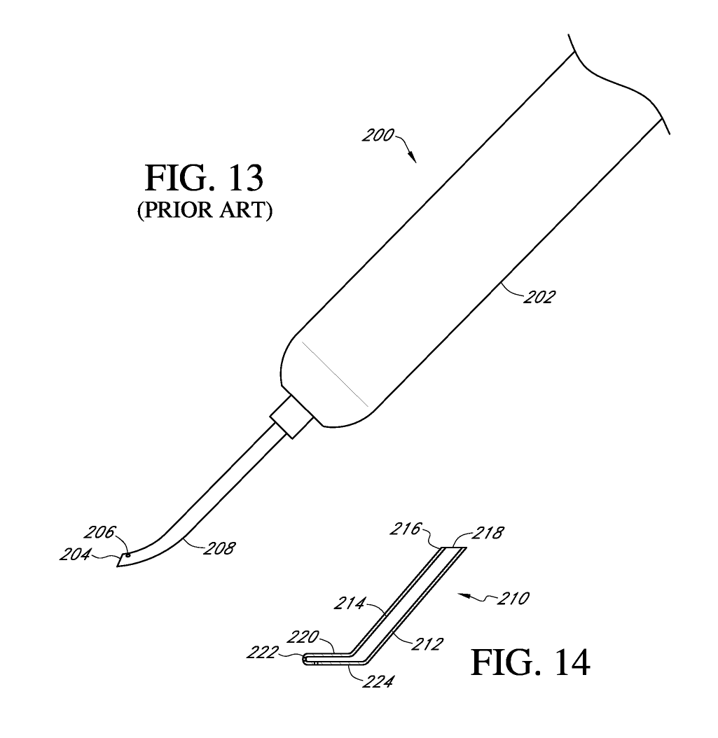

FIG. 13 illustrates a typical bimanual irrigation handpiece 200, and FIG. 14 illustrates another of the present embodiments configured for use with the handpiece 200 of FIG. 13. Some surgeons prefer to use bimanual irrigation and aspiration (separate handpieces for irrigation and aspiration) instead of the typical combined I/A probe for cortex and viscoelastic removal during cataract surgery. With reference to FIG. 13, the handpiece 200 comprises an elongate handle portion 202 having a distal end 204. An irrigation port 206 is located at the distal end 204. An irrigation lumen (not shown) extends through the handpiece 200 to the irrigation port 206 at the distal end 204. The distal end 204 includes an arcuate bend 208.

With reference to FIG. 14, the sleeve 210 includes a tubular portion 212 defining a passage 214 configured to receive the distal end 204 of the handpiece 200. The sleeve 210 further includes a flange 216 at its proximal end 218, a flat superior portion defining a haptic engagement surface 220 at its distal end 222, a convex inferior surface 224, and four irrigation ports (not shown) positioned about the haptic engagement surface 220, with one anterior port, one inferior port, and two lateral ports, as described above with respect to the foregoing embodiments. The sleeve 210 is thus configured to engage the IOL and to provide irrigation in a similar manner as described above with respect to the foregoing embodiments. The sleeve 210 is preferably constructed of a flexible and resilient medical grade material, such as any of the materials described above with respect to the foregoing sleeves. Similarly, the sleeve 210 may be single use (disposable) or reusable.

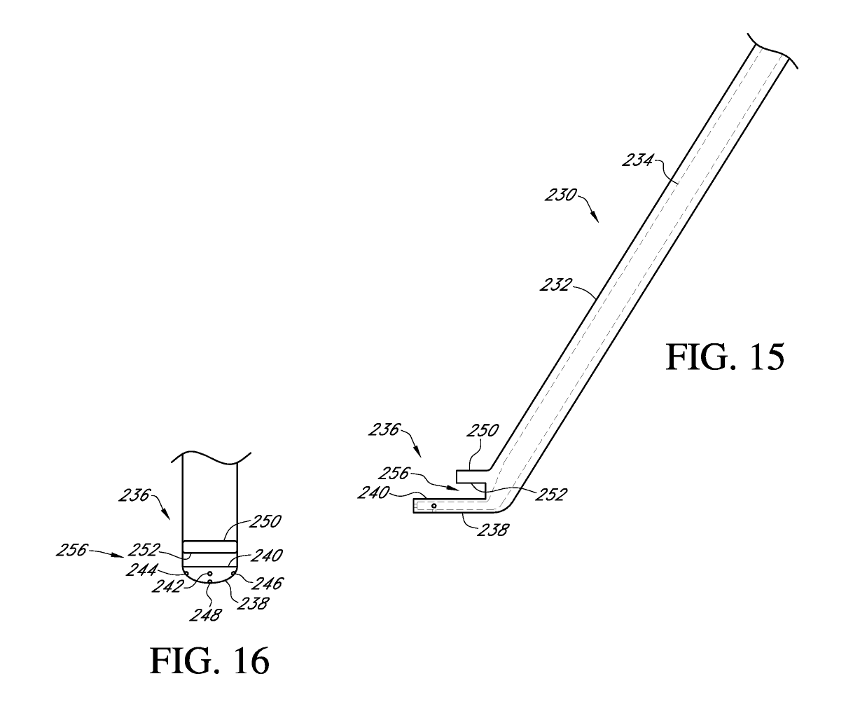

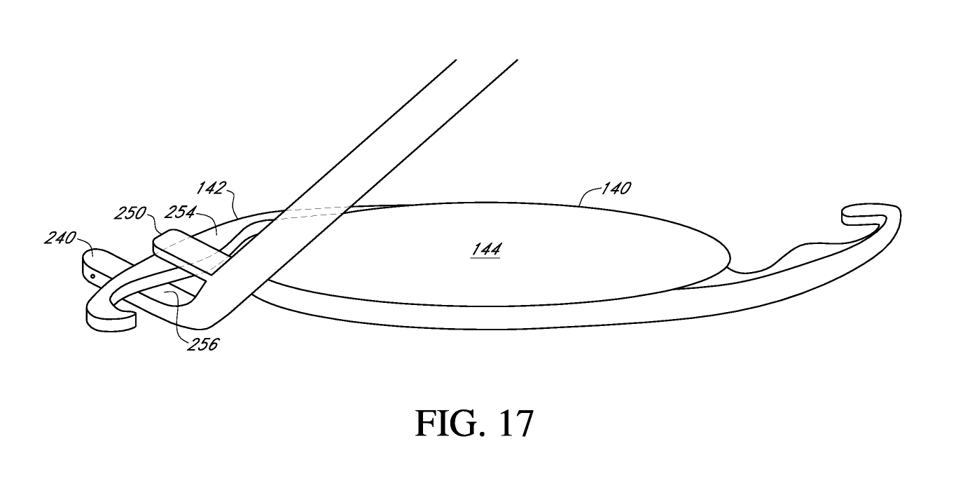

FIGS. 15 and 16 illustrate another embodiment of a handpiece 230 for positioning an intraocular lens (IOL) within the capsular bag of an eye. The handpiece 230 is similar to the embodiment shown in FIG. 2, and includes a tubular handle portion 232, an irrigation lumen 234, a lens engagement portion 236 with a convex inferior surface 238 and a flat superior surface defining a haptic engagement surface 240, four irrigation ports 242, 244, 246, 248 (FIG. 16) arranged about the haptic engagement surface 240 (one anterior 242, two lateral 244, 246, and one inferior 248), and no aspiration port. The lens engagement portion 236 of the handpiece 230 of FIGS. 15 and 16 further includes a haptic capture portion 250 spaced from the haptic engagement surface 240. The haptic capture portion 250 comprises a cantilevered projection or ledge extending laterally at the distal end of the handpiece 230 superior to the haptic engagement surface 240. The haptic capture portion 250 includes a flat inferior surface 252 that is parallel to the haptic engagement surface 240. With reference to FIG. 17, the haptic capture portion 250 is configured to engage an upper surface 254 of the IOL haptic 142 during a procedure for rotating the IOL 140. The haptic 142 is received in the space 256 defined between the haptic engagement surface 240 and the inferior surface 252 of the haptic capture portion 250. With the haptic 142 received in the space 256, the operator is able to exercise greater control over the IOL 140, because force is applied to the haptic 142 both from above and below. In one or more alternative embodiments, the inferior surface of the haptic capture portion need not be parallel to the haptic engagement surface. For example, the inferior surface of the haptic capture portion may be angled to define a variable spacing between the inferior surface of the haptic capture portion and the haptic engagement surface.

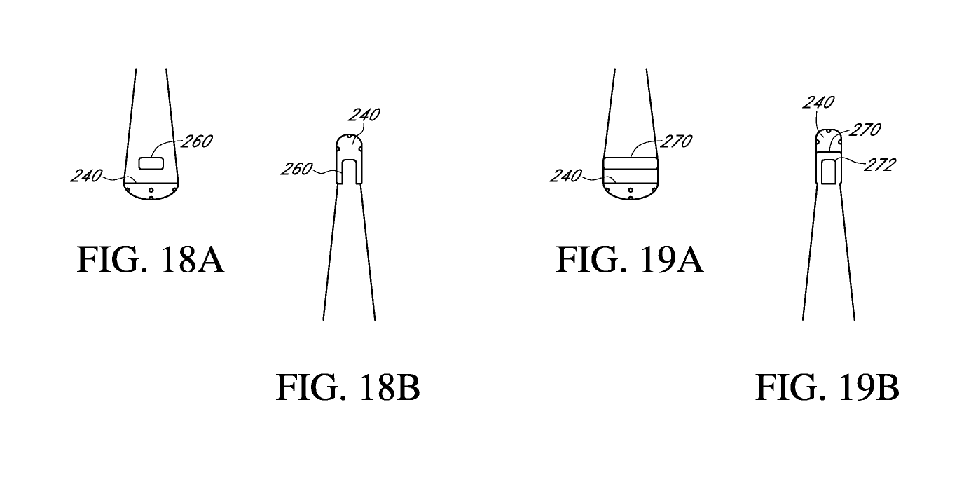

In the embodiment of FIGS. 15 and 16, a width of the haptic capture portion 250 is substantially equal to a width of the haptic engagement surface 240 (FIG. 16). FIGS. 18A and 18B illustrate an alternative embodiment in which a width of the haptic capture portion 260 is less than the width of the haptic engagement surface 240. The narrower haptic capture portion 260 advantageously enables greater visualization of the IOL 140 during the implantation procedure, since the operator's eyes are located above the haptic capture portion 260 of the handpiece. FIGS. 19A and 19B illustrate yet another alternative embodiment in which the haptic capture portion 270 includes a central opening 272 (FIG. 19B). The opening 272 also advantageously enables greater visualization of the IOL 140 during the implantation procedure, since the operator can see through the opening 272 from above.

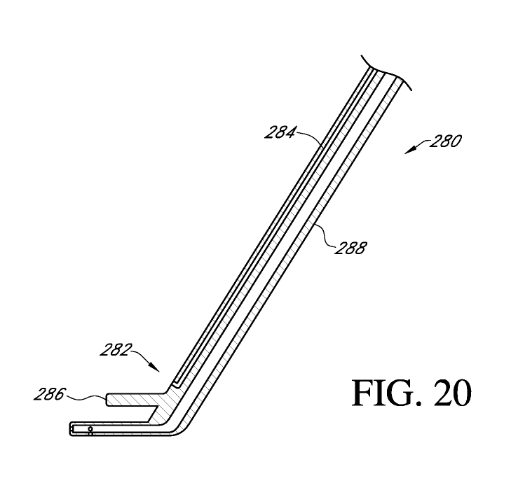

FIG. 20 illustrates another embodiment of a handpiece 280 for positioning an intraocular lens (IOL) within the capsular bag of an eye. The handpiece 280 is similar to the embodiment shown in FIG. 15, except that an aspiration port 282 and an aspiration lumen 284 are also provided. The aspiration port 282 is located just proximal of the haptic capture portion 286, and the aspiration lumen 284 extends through the handle portion 288 of the handpiece 280 to the aspiration port 282. The embodiment of FIG. 20 enables both irrigation and aspiration, and achieves the advantages of each described above with respect to the foregoing embodiments.

Any of the embodiments described above with respect to FIGS. 15-20 may be adapted for a sleeve. For example, FIGS. 8, 9, 11, 12, and 14 illustrate sleeve embodiments that may be modified to include any or all of the features described with respect to FIGS. 15-20, including adding a haptic capture portion and/or an aspiration port.

As described above, the present embodiments advantageously facilitate rotation of intraocular lenses after implantation with a decreased likelihood of tearing the capsular bag. Any of the embodiments described above may also be adapted for use without irrigation or aspiration. For example, no irrigation ports or aspiration ports may be provided. Such an embodiment may comprise a device configured for use with an alternative source of irrigation fluid, or with a viscoelastic component providing spacing that is advantageous for IOL rotation. This alternative device may be constructed of a solid piece of metal and/or polymer, for example, and may be simply a handheld mechanical device that is used to rotate an IOL.

The above description presents various embodiments of the present invention, and the manner and process of making and using them, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention.

1-20. (canceled)

21. Apparatus for positioning an intraocular lens (IOL) having a haptic within the capsular bag of an eye, the apparatus comprising:
a handle portion having a proximal end, a distal end, and an irrigation lumen extending between the proximal end and the distal end; and
a lens engagement portion at the distal end of the handle portion, the lens engagement portion comprising:
a superior surface defining a haptic engagement surface configured to engage the haptic of the IOL;
a haptic capture portion spaced from the haptic engagement surface; and
a plurality of irrigation ports arranged about the haptic engagement surface, each of the irrigation ports being in fluid communication with the irrigation lumen.

22. The apparatus of claim 21, wherein the irrigation ports comprise an anterior port, a pair of lateral ports, and an inferior port.

23. The apparatus of claim 21, wherein the haptic capture portion includes an inferior surface parallel to and spaced from the haptic engagement surface.

24. The apparatus of claim 21, wherein the haptic capture portion has a first width and the haptic engagement surface has second width greater than the first width.

25. The apparatus of claim 21, wherein the haptic capture portion includes an opening configured and located to enable visualization of the IOL from above.

26. The apparatus of claim 21, wherein the lens engagement portion has a convex inferior surface.

27. The apparatus of claim 26, wherein a radius of the inferior surface is less than or equal to a maximum perpendicular distance between the inferior surface and the haptic engagement surface.

28. The apparatus of claim 26, wherein a radius of the inferior surface is greater than a maximum perpendicular distance between the inferior surface and the haptic engagement surface.

29. The apparatus of claim 21, wherein the handle portion and the lens engagement portion define an angle between approximately 5° and approximately 50°.

30. An intraocular lens (IOL) positioning sleeve configured for installation on a distal tip portion of an irrigation/aspiration handpiece having a handle with an irrigation lumen communicating with at least one handpiece irrigation port in the distal tip portion, wherein the distal tip portion defines an angle with respect to the handle, the IOL positioning sleeve comprising:
a tubular portion defining a passage configured to receive the distal tip portion;
a haptic engagement portion extending distally from the tubular portion and defining a haptic engagement surface configured for engaging a haptic of the IOL;
a shoulder between the tubular portion and the haptic engagement portion, wherein the shoulder is configured to be engaged by the distal tip portion of the handpiece when the distal tip portion is received in the passage; and
a plurality of sleeve irrigation ports in the haptic engagement portion, each of the sleeve irrigation ports being in fluid communication with a handpiece irrigation port.

31. The IOL positioning sleeve of claim 30, wherein the sleeve irrigation ports comprise an anterior port, a pair of lateral ports, and an inferior port.

32. The IOL positioning sleeve of claim 30, wherein the haptic engagement portion includes a convex inferior surface.

33. The IOL positioning sleeve of claim 30, wherein the distal tip portion of the hand-piece defines an angle Θ with the handle of the handpiece, and wherein the tubular portion of the sleeve is configured with a bend having an angle substantially conforming to the angle Θ.

34. The IOL positioning sleeve of claim 30, wherein the tubular portion has a first diameter and the haptic engagement portion has a thickness less than the first diameter.
